(19) United States
(12) Patent Application Publication
(10) Pub. No.: US 2010/0292474 A1
(43) Pub. Date: Nov. 18, 2010

(54) DI-ALKYL SUBSTITUTED IMIDAZOLINE DERIVATIVES

(76) Inventor: Hugo Jean Marie Demeyere, Merchtem (BE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/779,071

(22) Filed: May 13, 2010

(30) Foreign Application Priority Data
May 14, 2009 (EP) 09160280.5

(51) Int. Cl. C07D 239/20; C07D 233/64

(52) U.S. Cl. 544/335; 548/341.5

(57) ABSTRACT

The present invention describes a granular fabric softening composition comprising a di-alkyl substituted imidazoline derivative according to the formula;

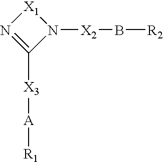

wherein;
X₁ is a C2-3 alkylene group;
X₂ and X₃ are independently C1-6 linear or branched alkylene or alkenylene groups;
R₁ and R₂ are independently C8-22 linear or branched alkyl or alkenyl groups;
characterized in that;
A and B are independently O—(C═O)—.

### FIELD OF THE INVENTION

The present invention relates to di-alkyl substituted imidazoline derivatives where the two alkyl groups are linked through an ester function to the imidazoline core. The di-alkyl substituted imidazoline derivatives of the present invention may also be used in compositions such as fabric care compositions and hair conditioning compositions.

### BACKGROUND TO THE INVENTION

Supply and storage of fabric softening compositions and their individual ingredients in less developed countries can pose logistical problems. Oftentimes, the transportation of products is over long distances, using poor infrastructures, and in high temperatures. Also, due to limitations to resources in less developed countries, it is preferential that the used fabric conditioning compositions are readily biodegradable and have minimal environmental impact.

Traditionally, fabric softening compositions have used dialkyl quaternary conditioning actives. However, a major drawback to these compounds is that they need to be stored and shipped with the addition of a dilution solvent. Considering the logistical issues associated with storage and shipping in less developed countries, it is preferable to not use solvents.

Di-alkyl substituted imidazoline compounds are known in the art. Oftentimes they are used as fabric softening and hair conditioning actives. However, other industrial applications include the use as antistatic and as corrosion inhibitor. Given their lower melting point, the di-alkyl substituted imidazoline derivatives can be stored and shipped without the need to add a dilution solvent, leading to important logistical cost savings. They are also non-corrosive, meaning construction material of lower grade metallurgy can be utilised for manufacturing, storage and shipment equipment. Nevertheless, di-alkyl substituted imidazoline derivatives are not readily biodegradable, which is an undesirable characteristic of this class of compounds. EP 345,842 A2 discloses di-alkyl substituted imidazoline derivatives which exhibit improved biodegradability; however, these have since been found to form persistent monoalkyl substituted break-down products. Therefore, only partial biodegradation can be achieved which is environmentally undesirable. However, JP05-105670 and JP06-228870 describe readily biodegradable di-alkyl imidazole derivatives.

There is a need in the art for fabric softening compositions that comprise readily biodegradable actives which do not form persistent monoalkyl substituted break-down products upon degradation. There is also a need for a fabric softening composition in which the fabric softening active can be easily and safely stored and transported over long distances using poor infrastructures and in high temperatures. Lastly there is a need for a fabric softening composition for use in less developed countries that comprises all of the above characteristics, but which is also in a consumer accepted product form.

It was surprisingly found that granular fabric softening compositions comprising a di-alkyl substituted imidazole derivative according to the formula of claim 1 offered a fabric softening composition in which the softening active is readily biodegradable, and is in a consumer accepted product form. Also, the fabric softening active can be shipped safely and easily, in the absence of a dilution solvent.

### SUMMARY OF THE INVENTION

A first aspect of the present invention is a granular fabric softening composition comprising a di-alkyl substituted imidazoline derivative according to the formula;

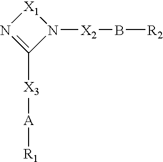

wherein;
X₁ is a C2-3 alkylene group;
X₂ and X₃ are independently C1-6 linear or branched alkylene or alkenylene groups;
R₁ and R₂ are independently C8-22 linear or branched alkyl or alkenyl groups;
characterized in that;
A and B are independently O—(C═O)—

### DETAILED DESCRIPTION OF THE INVENTION

The present invention is to a granular fabric softening composition comprising a di-alkyl substituted imidazoline derivative according to the formula;

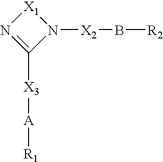

wherein, X₁ is a C2-3 alkylene group, X₂ and X₃ are independently C1-6 linear or branched alkylene or alkenylene groups, R₁ and R₂ are independently C8-22 linear or branched alkyl or alkenyl groups, A and B are independently selected from the group comprising O—(C═O)—, —(C═O)—O—, or mixtures thereof. Preferably, A and B are each independently of the formula O—(C═O)—. In a preferred embodiment, X₁ is a C2 alkyl group. In another preferred embodiment, X₂ is a C2-3 alkylene group, more preferably a C2 alkylene group. In another preferred embodiment, X₂ is an isopropyl group. In a further preferred embodiment, X₃ is a C1-4 alkylene group, more preferably, a CH₂ group. In yet another preferred embodiment, R₁ and R₂ are independently a C12-18 alkyl or a C12-18 alkenyl group. Preferably, R₁ and R₂ are independently selected from the group comprising (hydrogenated) tallow fatty acid, palm fatty acid, hydrogenated palm fatty acid, oleic acid, rapeseed fatty acid, hydrogenated rapeseed fatty acid or mixtures thereof. These di-alkyl substituted imidazoline derivatives can also be called imidazoline diester compounds.

Groups A and B, both independently being O—(C═O)—, act as points of degradation, ensuring the diester derivative does not form a persistent monoalkyl species. The formation of persistent monoalkyl substituted imidazoline species is avoided by having two weak ester bonds, such that an intermediate of the formula I or II will be degraded in the environment to form III.

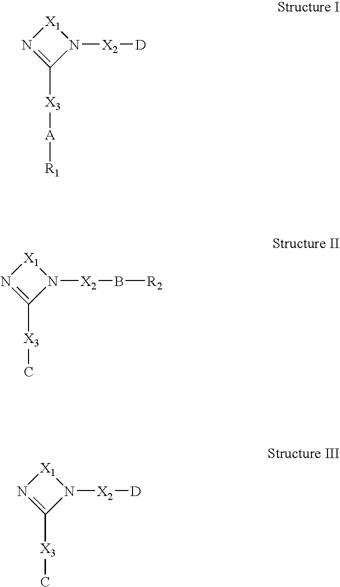

In the granular fabric softening compositions of the present invention, A and B are both independently of the formula O—(C═O)— as this structure is preferential for the synthesis process as described herein. However, those skilled in the art will recognize other suitable ester arrangements.

The granular fabric softening composition can be used as an additive in a hand or machine rinse or can alternatively be used in a tumble drier application. The granular composition can be dispersed in water during or prior to use.

The di-alkyl substituted imidazoline derivative is preferably present at a level from about 70% to about 99% by weight of the granular fabric softening composition.

Fabric softener compositions comprising the di-alkyl substituted imidazoline derivative may optionally comprise other ingredients, preferably selected from the group comprising quaternary ammonium compounds, silicones compound, perfumes, encapsulated perfumes, dispersing agents, stabilizers, pH control agents, colorants, brighteners, dyes, odor control agent, pro-perfumes, cyclodextrin, solvents, soil release polymers, preservatives, antimicrobial agents, chlorine scavengers, anti-shrinkage agents, fabric crisping agents, spotting agents, anti-oxidants, anti-corrosion agents, bodying agents, drape and form control agents, smoothness agents, static control agents, wrinkle control agents, sanitization agents, disinfecting agents, germ control agents, mold control agents, mildew control agents, antiviral agents, anti-microbials, drying agents, stain resistance agents, soil release agents, malodor control agents, fabric refreshing agents, chlorine bleach odor control agents, dye fixatives, dye transfer inhibitors, color maintenance agents, color restoration/rejuvenation agents, anti-fading agents, whiteness enhancers, anti-abrasion agents, wear resistance agents, fabric integrity agents, anti-wear agents, defoamers and anti-foaming agents, rinse aids, UV protection agents, sun fade inhibitors, insect repellents, anti-allergenic agents, enzymes, flame retardants, water proofing agents, fabric comfort agents, water conditioning agents, shrinkage resistance agents, stretch resistance agents, thickeners, chelants, electrolytes and mixtures thereof.

Another aspect of the present invention is a method of synthesizing the di-alkyl substituted imidazoline derivative.

A preferred synthesis method for making di-alkyl substituted imidazoline derivatives is as follows.

In a first step, a hydroxyl substituted polyalkylenediamine of the formula NH₂—X₁—NH—X₂—OH is reacted with a hydroxyl substituted fatty acid of the formula OH—X₃—COOH. In a preferred embodiment, the hydroxyl substituted polyalkylenediamine is N-(2-hydroxyethyl)-1,2-ethylenediamine. In another preferred embodiment, the hydroxyl substituted polyalkylenediamine is N-(2-hydroxyispropyl)-1,2-ethylenediamine. In another preferred embodiment, the hydroxyl substituted fatty acid is glycolic acid. In this first step, the hydroxyl substituted polyalkylenediamine and the hydroxyl substituted fatty acid are mixed in the molten phase, and the reaction mixture is kept agitated and in the liquid phase. The molar ratio of hydroxyl substituted fatty acid to hydroxyl substituted polyalkylenediamine is preferably between about 0.8:1 to about 1.2:1, more preferably between about 0.9:1 to about 1:1. Preferably, the reaction vessel is kept under an inert atmosphere, for example, by flushing it with N₂ gas.

In a second step, the reaction mixture of the first step is heated to a temperature of from about 100° C. to about 240° C., preferably from about 150° C. to about 210° C., most preferably from about 180° C. to about 200° C. The reaction water is then removed from the reactor vessel, preferably by applying a vacuum. The resultant reaction mixture will then contain mainly a di-hydroxy substituted imidazoline of the formula;

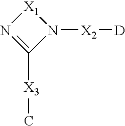

wherein X₁ is a C2-3 alkylene group, X₂ is a C1-6 linear or branched alkylene or alkenylene group, X₃ is a C1-6 linear or branched alkylene or alkenylene group and C is OH and D is OH. Preferably, X₁ is CH2-CH2, X₂ is CH2-CH2 or CH—(CH3)-CH2, X₃ is CH2 and C is OH and D is OH.

In a third step, the di-hydroxy substituted imidazoline of step 2 is further reacted with an esterifying agent. The esterifying agent is preferably selected from the group comprising fatty acids, short chain, C1-4, esters of fatty acids and fatty acid glycerides. The molar ratio of esterifying agent to di-hydroxy substituted imidazoline is from about 1.2:1 to about 2.2:1, preferably from about 1.5:1 to about 2:1. The reaction mixture is heated to a temperature from about 100° C. to about 200° C., preferably from about 165° C. to about 190° C. The reaction water is then removed from the reactor vessel, preferably by applying a vacuum. The reaction is preferably kept under an inert atmosphere, at a pressure less than about 1500 Pa, preferably less than about 250 Pa. When the esterifying agent is a fatty acid ester or fatty acid glyceride, preferably, an esterification catalyst is used. The esterification catalyst is preferably selected from the group comprising metal alkoxides, metal halides, metal alkyl halides, metal carboxylate salts, metal alkyl alkoxides and mixtures thereof. In one embodiment the esterification catalyst is a metal alkoxide, preferably selected from the group comprising Sn(OR)4, Na(OR), K(OR) or Ti(OR)4, wherein R is a C1-6 alkyl group, preferably C2-3. In another embodiment, the esterification catalyst is a metal halide, preferably selected from the group comprising tin halides or transition metal halides, preferably TiY4 where Y is Cl or Br. In a further embodiment, the esterification catalyst is a metal alkyl halide, preferably a transition metal alkyl halide. In yet another embodiment, the esterification catalyst is a metal carboxylate salt, preferably selected from the group comprising tin acetate or zinc acetate. In a further embodiment, the esterification catalyst is a metal alkyl alkoxide, preferably selected from the group comprising transition metal alkyl alkoxides or tin alkyls such as SnT4 where T is independently a C1-8 hydrocarbyl group or a C1-8 hydroxyl group.

### EXAMPLES

Preferred di-alkyl substituted imidazoline derivatives of the present invention include;
a) Reaction product of N-(2-hydroxyethyl)-1,2-ethylenediamine with glycolic acid, esterified with fatty acid, where the fatty acid is (hydrogenated) tallow fatty acid, palm fatty acid, hydrogenated palm fatty acid, oleic acid, rapeseed fatty acid, hydrogenated rapeseed fatty acid or a mixture of the above.
b) Reaction product of N-(2-hydroxyisopropyl)-1,2-ethylenediamine with glycolic acid, esterified with fatty acid, where the fatty acid is hydrogenated tallow fatty acid, palm fatty acid, hydrogenated palm fatty acid, oleic acid, rapeseed fatty acid, hydrogenated rapeseed fatty acid or a mixture of the above.
c) Reaction product of N-(2-hydroxyethyl)-1,2-ethylenediamine with glycolic acid, esterified with the methyl ester of hydrogenated tallow fatty acid, palm fatty acid, hydrogenated palm fatty acid, oleic acid, rapeseed fatty acid, hydrogenated rapeseed fatty acid or a mixture of the above.
d) Reaction product of N-(2-hydroxyisopropyl)-1,2-ethylenediamine with glycolic acid, esterified with the methyl ester of hydrogenated tallow fatty acid, palm fatty acid, hydrogenated palm fatty acid, oleic acid, rapeseed fatty acid, hydrogenated rapeseed fatty acid or a mixture of the above.
e) Reaction product of N-(2-hydroxyethyl)-1,2-ethylenediamine with glycolic acid, esterified with triglyceride or partial glyceride of (hydrogenated) fatty acids, such as tallow, palm, rapeseed etc.
f) Reaction product of N-(2-hydroxyisopropyl)-1,2-ethylenediamine with glycolic acid, esterified with triglyceride or partial glyceride of (hydrogenated) fatty acids, such as tallow, palm, rapeseed etc.

Preferred compositions comprising di-alkyl imidazoline derivatives include;

| | Examples | | | | | | |
|---|---|---|---|---|---|---|---|
| INGREDIENTS | I | II | III | IV | V | VI | VII |
| Imidazoline Di- | 75% | 85% | 95% | | | | |
| Ester ^{a} | | | | | | | |
| Imidazoline Di- | | | | 75% | | 85% | |
| Ester ^{b} | | | | | | | |
| Imidazoline Di- | | | | | 85% | | 90% |
| Ester ^{c} | | | | | | | |
| Nonionic surfactant ^{d} | 10% | 8% | 3% | 12% | 3% | 10% | 5% |
| Citric acid ^{e} | 7% | — | | 5% | 5% | | |
| Perfume | 0.30% | 1.50% | 1.00% | 1.00% | 1.00% | 1.20% | 0.25% |
| PEG 4000 ^{f} | 5% | 3% | — | 5% | 3% | | 3% |
| Blue Dye ^{g} | 0.01% | 0.030% | 0.030% | 0.030% | 0.030% | 0.030% | 0.01% |
| Deionized water | Balance | Balance | Balance | Balance | Balance | Balance | Balance |

Percentages are expressed as percentage by weight of the composition.
^{a} Imidazoline diester; reaction product of N-(2-hydroxyethyl)-1,2-ethylenediamine with glycolic acid, esterified with partially hardened tallow fatty acid.
^{b} Imidazoline diester; reaction product of N-(2-hydroxyethyl)-1,2-ethylenediamine with glycolic acid, esterified with fully hardened tallow fatty acid.
^{c} Imidazoline diester; reaction product of N-(2-hydroxyisopropyl)-1,2-ethylenediamine with glycolic acid, esterified with partially hardened palm fatty acid.
^{d} Nonionic surfactant C12-14 alkyl, 10 Ethoxylate
^{e} Anhydrous citric acid
^{f} Poly ethylene glycol MW 4000
^{g} Liquitint Blue AF from the Milliken company

The above products are produced by spray-chilling a co-melt of the imidazoline compound with the nonionic surfactant. The atomized co-melt is agglomerated and perfume (and eventually dye solution, citric acid and other minors) are sprayed on during agglomeration.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A granular fabric softening composition comprising a di-alkyl substituted imidazoline derivative according to the formula; 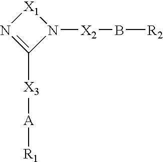
wherein;
X₁ is a C2-3 alkylene group;
X₂ and X₃ are independently C1-6 linear or branched alkylene or alkenylene groups;
R₁ and R₂ are independently C8-22 linear or branched alkyl or alkenyl groups;
characterized in that;
A and B are independently O—(C═O)—.

2. The granular fabric softening composition of claim 1, wherein X₁ of the di-alkyl substituted imidazoline derivative is a C2 alkylene group.

3. The granular fabric softening composition of claim 1, wherein X₂ of the di-alkyl substituted imidazoline is a C2-3 alkylene group, preferably a C2 alkylene group, more preferably an isopropyl group.

4. The granular fabric softening composition of claim 1, wherein X₃ of the di-alkyl substituted imidazoline is a C1-4 alkylene group, preferably, a CH₂ group.

5. The granular fabric softening composition of claim 1, wherein R₁ and R₂ of the di-alkyl substituted imidazoline are independently a C12-18 alkyl or a C12-18 alkenyl group, more preferably independently selected from the group comprising (hydrogenated) tallow fatty acid, palm fatty acid, hydrogenated palm fatty acid, oleic acid, rapeseed fatty acid, hydrogenated rapeseed fatty acid or mixtures thereof.

6. The granular fabric softening composition of claim 1, wherein the di-alkyl substituted imidazoline derivative is present at a level from about 70% to about 99% by weight of the granular composition.
